(19) United States
(12) Patent Application Publication
(10) Pub. No.: US 2009/0306767 A1
(43) Pub. Date: Dec. 10, 2009

(54) SHAPE MEMORY DEVICES

(75) Inventor: Andreas Lendlein, Berlin (DE)

Correspondence Address: HOGAN & HARTSON LLP;IP GROUP, COLUMBIA SQUARE, 555 THIRTEENTH STREET, N.W., WASHINGTON, DC, 20004, US

(73) Assignee: MNEMOSCIENCE GMBH, Übach-Palenberg (DE)

(21) Appl. No.: 12/299,739

(22) PCT Filed: May 7, 2007
(86) PCT No.: PCT/EP2007/004010

§ 371 Date: Dec. 16, 2008

(30) Foreign Application Priority Data
May 5, 2006 (EP) 06009369.7

(51) Int. Cl. A61F 2/06; C08L 23/00; B29C 63/34

(52) U.S. Cl. 623/1.18; 525/55

(57) ABSTRACT

The present invention concerns shape memory devices, methods of producing same and the use of these materials.

The present invention concerns shape memory devices, methods of producing same and the use of these materials.

### PRIOR ART

Shape memory polymers are an interesting class of materials, which have received considerable attention in recent years. Shape memory functionality is the ability of a material to temporarily fix a second shape after an elastic deformation and only recover its original shape after application of an external stimulus. While this effect is one-way, reversible shape changes induced by cooling and heating, i.e. a two-way effect may also be realized.

The advantageous and intriguing properties of these materials are in particular the possibility to initiate a desired change in shape by an appropriate external stimulus, so that an original shape, after deformation, is re-established, and the possibility to deform and program these materials so that highly specific configurations and shape changes can be obtained. The deformed shape is often called the temporary shape in the art. The phenomenon is a functionality and not an inherent material property. The effect/functionality results from a combination of polymer structure and specific functionalization processes.

The first materials known to provide this functionality were metallic shape memory alloys. In the recent past, shape memory polymers have been developed. Typical shape memory polymers are, for example, phase segregated linear block copolymers, having a hard segment and a switching (soft) segment. Important representatives of these types of materials are disclosed in the international publications WO 99/42147 and WO 99/42528. These materials employ as external stimulus for initiating the recovery of the original shape a change in temperature, usually a temperature rise. Shape memory polymers being susceptible to other external stimuli are also known, such as the photosensitive shape memory polymers disclosed in WO 2004/062706.

For some applications it would however be desirable to have a material which displays a shape memory effect in response to the exertion of mechanical forces (tensile stress, compression load etc), a stimulus towards which conventional shape memory polymers do not react by showing a shape memory effect (rather conventional shape memory polymers would show initially no response and finally, with strong mechanical forces, would show mechanical failure).

Accordingly the present invention aims at providing a material or composition or device which enables the selective initiation of a shape memory effect in response to the external stimulus “mechanical force”.

### BRIEF DESCRIPTION OF THE INVENTION

The present invention solves the above object with the shape memory device as defined in claim 1. Preferred embodiments are outlined in claims 2 to 7. Furthermore the present invention provides the method for preparing such shape memory devices as outlined in claim 8 as well as the use as defined in claims 9 and 10.

### DESCRIPTION OF THE FIGURES

FIG. 1 shows the use of a first material in sheet form which is then folded or rolled into a deformed shape. This deformed shape is then fixed by a second material, shown in FIG. 1 in the shape of a tube. After application of the suitable external stimulus the first material recovers the original non deformed shape.

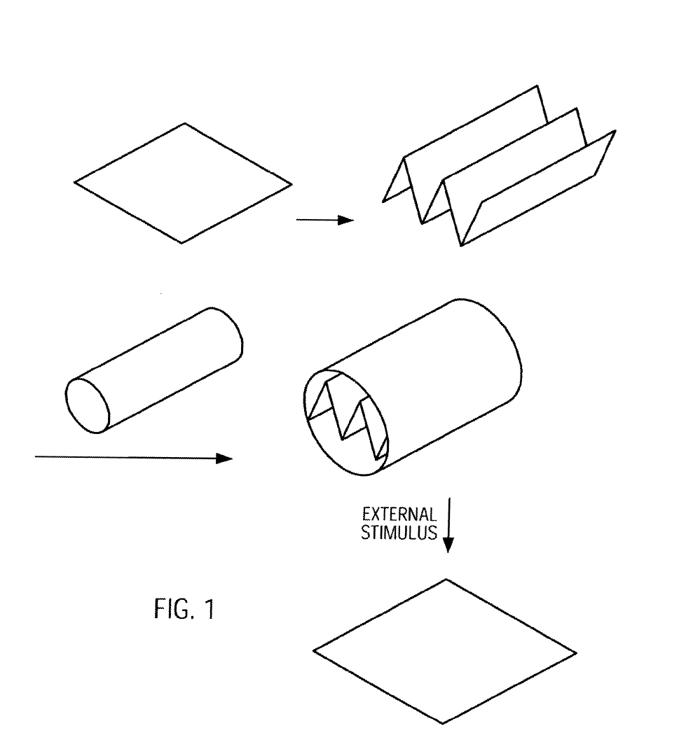

FIG. 2 shows the use of the first material in the form of a tube, such as a stent or the like. This tube is then deformed by compressing and the deformed shape is fixed by a coating of the second material (FIG. 2a) or the use of fibers of the second material, which are wrapped around the article formed from the first material (FIG. 2b). After application of the suitable external stimulus the first material recovers the original non deformed shape.

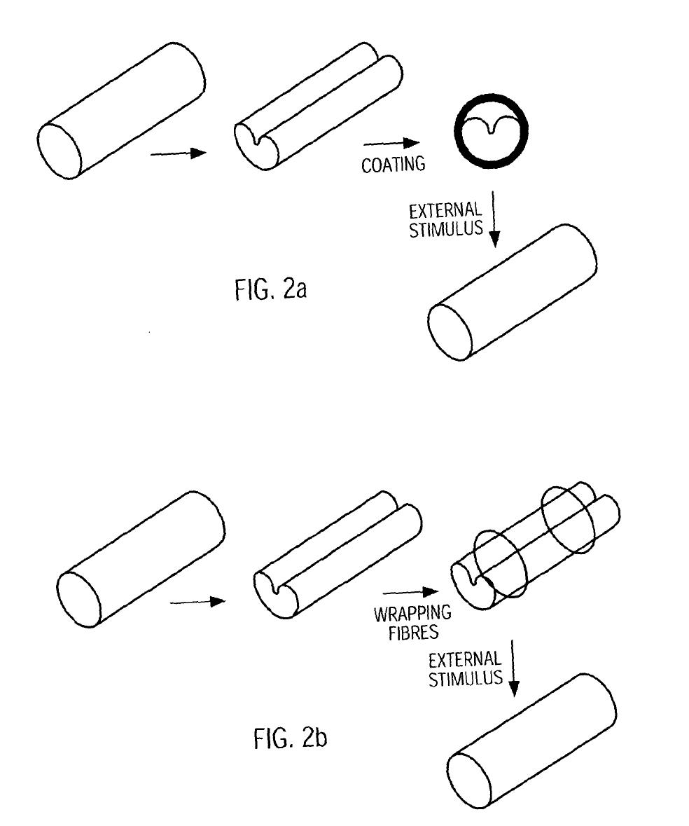

### DETAILED DESCRIPTION OF THE PRESENT INVENTION

As defined in claim 1, the shape memory devices of the present invention are sensitive towards an external stimulus, in particular mechanical forces, i.e. they show a shape memory effect after having been subjected to mechanical forces. As is further derivable from claim 1 the device comprises a first material which can memorize at least one shape, i.e. in accordance with the usual designation in the art the permanent shape. This material, in the device, is present in the deformed, i.e. temporary shape. However, in accordance with the present invention it is not required that the deformed state, i.e. the temporary shape (the designation as used again corresponds to the usual designation employed in the art for shape memory polymers) is fixed by means of interactions within the material (for example by chemical or physical interactions of soft or switching segments of shape memory polymers) since the device in accordance with the present invention comprises an additional second material fixing the deformed shape. In this respect the present invention in particular requires that the first material is elastic, in order to allow a recovery of the permanent shape after release of the fixation provided by the second material.

Preferred first materials are accordingly elastomeric polymer networks, having a low glass transition temperature and being highly flexible. Further examples of preferred first materials are shape memory polymers with a switching temperature below the temperature at which the shape memory device in accordance with the present invention is to be used.

The device in accordance with the present invention accordingly fixes the deformed shape by an additional second material which may for example be provided in the form of a coating, partially or completely covering the article made from the first material. This second material displays a sufficient mechanical strength and physical integrity so that the temporary shape is secured. However, the second material is selected so that the application of a suitable external stimulus leads to a decrease of the mechanical strength of the second material or to the removal, partially or completely of the second material, so that the deformed shape cannot be maintained anymore. Instead the first material, no longer fixed by the second material, recovers its initial permanent shape, i.e. the deformed shape is lost and the permanent shape is formed.

As indicated above a suitable external stimulus in particular is a mechanical manipulation, such as a compression or a tensile stress. The use of a compression as external stimulus may in particular be of advantage for devices in accordance with the present invention which are manipulated before use with tools or using hands allowing the quick and easy application of a compressing force. Examples are simple medical devices as well as sensors etc. which are to be activated etc. shortly before putting them into use. This type of external stimulus may in particular be used with fragile or brittle materials and/or materials having a predetermined breaking point or the like which can be easily be deformed/destroyed by applying a compressing force. A further alternative is the application of a solvent in which selectively only the second material may be dissolved or at least swollen, so that the mechanical fixation is removed. Such a swelling leads to the formation of a gel phase of the second material so the mechanical integrity required for fixing the first material in the deformed shape is no longer given. The second material may for example be selected from brittle materials which show a sufficient integrity and/or cohesion that the deformed shape can be fixed, even against the internal forces within the first material. When this second material however is subjected to a mechanical manipulation as mentioned above, the integrity and/or cohesion of the second material is disturbed so that the second material can no longer hold the first material in the deformed shape. In accordance with the present invention it is however not only envisaged to use second materials which are sensitive towards a compression or tensile load, i.e. mechanical forces, but also which loose, as outlined above, the required integrity and/or cohesion upon application of other stimuli, such as the solvent sensitive materials mentioned above, examples of which are polyethylene glycol and polyvinyl alcohol. Such solvent sensitive materials may in particular be of use for devices which shall detect the presence of such solvents in safety sensors etc. Other examples are medical devices which can be, immediately before use be placed into a container comprising the solvent towards which the materials is sensitive.

Examples of suitable second materials are materials which soften due to heating, so that the above described effect occurs after having subjected the device to a heat treatment. The softening according to the invention does also include liquidation, that may be followed by evaporation, and also sublimation. In a preferred embodiment of the invention the second material is in its solid or at least highly viscous state when fixing the first material and is liquid with a viscosity low enough to release the memory form of the first material. This phase or viscosity change of the second material can be achieved e.g. be heating, by mechanical or by chemical treatment, like e.g. shaking or treatment with ultrasonic waves that lower e.g. the viscosity of thixotropic substances, or exposure to chemicals like gases or liquids that reduce the melting point and/or viscosity of the second material.

As long as only a part of the second material is liquified and/or reduced in viscosity, that is small/little/unimportant enough so that the fixing ability of the second material is not damaged, said part can advantageously form a film around the device of the invention, that can act as a lubricant to facilitate the insertion of the device into a place of destination, should this be aimed at.

In particular if short response times and/or a sharp temperature dependency are aimed for, it is a preferred embodiment of the invention that the second material is in its solid state, preferably in its crystalline or semicrystalline state, and in particular in its crystalline state, when fixing the first material, and is liquified to release the memorized form of the first material.

For many applications it is preferred for cost and environmental reasons, that the second material is a water based material, that preferably can be in the form of a gel or can be frozen and/or kept cool to fix the temporary form. The memorized form of the first material can then be released e.g. by heating to or above the melting point of the water based second material or by e.g. mechanical forces before it is unfreezed. Water based material means in the context of the present invention a composition, that includes at least about 20 weight-%, preferably at least about 50 weight-% and in particular at least about 85 weight-% water and that can also be advantageously almost pure water. The water based material can include in dissolved or dispersed form ingredients like e.g. organic solvents, thickeners, gases, organic and inorganic substances. Using such ingredients alone or in any combination allows to finely adjust the response to the stimulus that weakens the fixation ability of the second material. In the weakened form the water based material preferably is pure water, a solution, a dispersion or a gel.

In particular for medical applications the water based material can be a material that is compatible to the organism, in particular a physiological sodium chloride solution or the like, that may be sterilized and may contain further active agents and/or drugs. If a device with a frozen temporary form, that is fixed by a frozen second material, is implanted into a part of the human or animal body, there is an adjustable time window until the permanent form will be released, that can be controlled by the freezing temperature, the composition of the water based material and its amount. In this embodiment the water based material is preferably at least partly surrounded by the first material for thermal insulation.

A further option are pH sensitive materials, i.e. materials which even disintegrate after having been subjected to a suitable change in pH value (in particular for devices which are used in liquid containing environments, e.g. in specific parts of the human or animal body like the acidic milieu of the stomach). Other examples are light sensitive second materials or materials which are susceptible towards a hydrolytic degradation or an enzymatic degradation. Suitable examples of light sensitive materials are light sensitive shape memory materials such as those disclosed in WO 2004/062706, incorporated herein by reference. Examples of materials susceptible towards degradation are known to the skilled person and such materials in particular may be advantageously be used in medical devices, where disintegration may be facilitated/mediated by body fluids. In particular if the degradation products are not harmful such an embodiment is of high advantage for use in the medical field. In accordance with the present invention it is only necessary that the second material is able to fix and secure the deformed shape and that the second material is susceptible towards an external stimulus so that the first material, after the second material has been subjected to such a stimulus, recovers the remembered, i.e. permanent shape. Typical representatives of such suitable materials for the second material to be employed in accordance with the present invention are thermoplastic polymers.

If a thermoplastic polymer is heated above a transitions temperature Tₜᵣₐₙₛ, that may be a glass transition temperature or a melting point, it becomes soft and principally capable of flowing. At this point or at a higher temperature it may loose its ability to fix the temporary form of a first material. The temperature increase may be induced directly by heating or by other energy sources like electromagnetic radiation that is absorbed, or by mechanical impact like rubbing or ultrasonic waves. For a more precise phase transition crystalline or semicrystalline thermoplastic polymers, that have a melting point, are preferred. The desired fixation power and transition temperature can be adjusted e.g. by the thickness of the second material including said thermoplastic polymer, the percentage it is in contact with the first material, its chemical composition and by blending of the thermoplastic polymer with one or several other polymers and/or with know polymer additives. In the context of the present invention, thermoplastic polymers are preferably selected from the polymers or copolymers listed in the following, or include at least a monomer therefrom: vinyl polymers, polyethylene (PE), low density polyethylene (LDPE), high density polyethylene (HDPE), polypropylene (PP), styrene polymers, polystyrene (PS), styrene-acryinitrile copolymer (SAN), styrene-butadiene-styrene copolymer (SBS), styrene-butadiene-crystallizable poly(ε-caprolactone) copolymer (SBC), styrene-crystallizable poly(ε-caprolactone) copolymer (SC), styrene-isoprene-styrene copolymer (SIS), styrene-ethylene-butylene-styrene copolymer (SEBS), acryinitrile-butadiene-styrene copolymer (ABS), butadiene-crystallizable poly(ε-caprolactone) copolymer (BC), poly(ε-caprolactone) (PCL), polycarbonate (PC), poly(tetramethylene carbonate), PC/ABS, poly(methyl methacrylate) (PMMA), polyacrylnitrile (PAN), polymethacrylnitrile (PMAN), polyvinylacetate (PVAc), polyvinylalkohol (PVA), polyvinylchloride (PVC), poly(vinylidene chloride) (PVDC), poly(vinylidene chloride) copolymer, polytetrafluorethylene (PTFE), polybutadiene, poly(dimethyl butadiene) polyoxymethylene (POM), polyester, poly(ethylene terephthalate) (PET), polydimethylsiloxane, polyamide (PA), celluloseester, cellulose acetate, cellulose acetate propionate, cellulose acetate butyrate, cellulose propionate, cellulose triacetate, polyurethanes, poly(ether esters), poly(ether amides), polyether, poly(phenylene oxide) (PPO), poly(propylene oxide), PPO/PS, poly(butylene terephthalate) (PBT), polysulfone (PSU), aromatic polyester (APE), polyamideimide (PAI), poly(ether imide) (PEI), poly(ether sulphone) (PES), poly(ether ether ketone) (PEEK), poly(phenylene sulfide) (PPS), ethylene-propylene-diene copolymer (EPDM), EPDM/PP, natural rubber-PP, polyethylene-vinyl acetate (EVA), EVA/PVDC, nitrile rubber/PP, or modifications or derivatives thereof.

The thermoplastic polymer can be selected by known physical and chemical data of the polymers and by usual experiments to best fit for the given application in terms of e.g. transition temperature; mechanical strength for fixation; processability; compatibility with the first material, e.g. that it can depending on the desired application be removed easily, or to the contrary that the adhesion is strong enough to remain on the first material for a second programming step; and/or compatibility with the surrounding in the given application, in particular biocompatibility and non-toxicity.

The first material to be employed in accordance with the present invention may be any material which is able to maintain at least one shape in memory, i.e. which is able to recover the original shape after a deformation (and the fixation of the deformed shape by means of the second material). Suitable examples thereof are shape memory polymers as for example illustrated in the prior art references mentioned above. However, as outlined above, any material which is able to remember one shape may be employed in accordance with the present invention. Accordingly the present invention also contemplates to use as first material, as already indicated above, elastic materials, in particular rubber materials of natural or synthetic origin. Also such elastic materials, such as natural or synthetic rubber, including EPDM materials and the like, are materials which, after an elastic deformation, display the ability to return to the non-deformed state after the external force (i.e. in the present invention the restraining coating of the second material) fixing the deformed shape is removed. Elastic materials according to the invention also include resilient, springy and superelastic materials and also include such metallic materials, alloys, composites, and even complex devices. Preferred in this respect are rubber materials in the form of polymer networks having main chain segments providing a domain having a rather low glass transition temperature. At the same time it is preferred when these materials display only a minimal hysterises.

One further possibility in this respect is the use of a shape memory polymer which has been programmed and accordingly provides one permanent shape and at least one temporary shape (depending on the number of switching segments) so that in addition to the permanent shape and the temporary shape as enabled by the shape memory polymer a further deformed shape is made possible. In this embodiment the temporary shape as programmed onto the shape memory polymer is deformed further and this additional deformed shape is then fixed in accordance with the present invention using the second material. Accordingly in such an embodiment the first shape change occurs when the second material is no longer able to secure the deformed shape—the shape memory polymer returns to the temporary shape from which the permanent shape may be recovered upon initiating the shape memory effect of the shape memory polymer.

By this embodiment it is easily and cost-efficiently possible to obtain a device with two shapes in memory, the permanent and the temporary shape, and further on the deformed shape (a so called triple-shape material). The same effect could be achieved with a device according to the invention including a first material with a permanent form that is deformed and fixed with a second material into a deformed shape and which is then deformed again and fixed with a different second material.

In addition it would be possible to again deform the deformed shape of a triple-shape material and fix this with a different second material to get a quad-shape material, and so on. Alternatively a triple-shape shape memory polymer with a permanent and two programmed temporary forms could be deformed and fixed accordingly. In this way very complex and flexible programming steps can be performed.

Depending on the intended use, the stimuli to switch the temporary to the permanent form and to release a deformed form, can be different or the same. If more than one deformed form is present, also the release of each deformed form can be effected by the same or different stimuli. In a preferred embodiment of the present invention at least one of said stimuli can be a predefined temperature and in particular all stimuli are differently predefined temperatures.

Usually the shapes of the triple-shape devices, quad-shape devices, et cetera, will be recovered in consecutive steps in the reverse order of the programming/deforming, but such devices can also be developed to optionally allow the direct switching of a “deeper” change in shape, e.g. the direct switching of the temporary to the permanent shape of a triple shape material, although the fixation by a second material is still effective. This is possible, if different stimuli are used and if the “deeper” change in shape is accompanied by mechanical forces strong enough to break or widen the second material(s) fixing the deformed form(s).

A triple-shape material is for example useful for fixation means, wherein the release of the temporary shape brings the means in the right position and/or shape and the permanent form secures the means by e.g. spreading, bending or curling. In a preferred embodiment of the present invention a triple-shape material as described above is used in the medical field, and particularly in a stent useful for non-invasive deployment, that is inserted in a compressed (deformed) shape, and that is expanded when a second material is triggered according to the invention to loose its fixing ability. This expanded form is then the therapeutic form of the stent. If the stent is to be removed from the patient because of a complication or because it is no longer needed, the smaller permanent form of the stent can be triggered by an appropriate stimulus to simplify the removal of the stent, e.g. with an appropriate catheter.

However, the present invention mainly is concerned with the fixation of a deformed state of a first material, which is able to memorize the original shape, using the second material, so that the device is able to recover this original shape when the second material no longer secures the deformed shape.

As indicated above, the second material partially or completely covers or at least fixes the article formed from the first material in the deformed shape. The type of coating, such as coating pattern, coating thickness etc. depends from the desired end use and the type of the first materials as well as the type of article and the degree of deformation. Complete coatings might in particular be required when the first material displays a strong tendency to recovering the original shape, while partial coatings may be suitable in particular in fields of application where the second material is rather expensive so that only the minimum required amount is to be used. However, these illustrative explanations shall not be construed as limitation since the skilled person will be in a position to determine the appropriate type of coating for the desired use and selected composition. A further alternative is the use of fibers or bands of the second material as well as sheets thereof which are used to be wrapped around the article formed from the first material being in the deformed shape. Thereby the deformed shape may be fixed as well without providing a coating of the second material onto the article formed from the first material.

As outlined above the second material may be selected from suitable materials providing a desired sensitivity towards an external stimulus. The following options are in particular envisaged by the present invention:

1.) thermo-sensitive materials application of heat softens the second material so that first material returns to permanent shape, i.e. non-deformed shape
2.) light-sensitive materials application of light softens or degrades the second material so that first material returns to permanent shape, i.e. non-deformed shape
3.) solvent-sensitive materials application of solvent selectively softens or removes the second material so that first material returns to permanent shape, i.e. non-deformed shape
4.) pH-sensitive materials variation of pH-value softens, degrades or removes the second material so that first material returns to permanent shape, i.e. non-deformed shape
5.) materials sensitive towards a magnetic field application of magnetic field softens the second material so that first material returns to permanent shape, i.e. non-deformed shape

According to the present invention sensitivity towards a magnetic field includes sensitivity towards an electromagnetic field and can be achieved by known means. Preferably said sensitivity is realized by the incorporation of magnetic particles, that may be fine particles with a diameter below 10 μm, nano particles with a diameter below 400 nm, but also coarse particles with a diameter above 10 μm and also filaments, wherein a magnetic field can by absorbed to generate heat as described e.g. in U.S. Pat. No. 6,991,698, U.S. Pat. No. 6,992,155, or U.S. Pat. No. 6,054,210, that are incorporated herein in their entirety.

The second material furthermore may be selected to have suitable properties, such as biocompatible materials, erodible materials, materials which degrade, for example by hydrolytic or enzymatic processes, crystalline, semicrystalline or amorphous materials and the like, depending in particular from the desired end use of the device formed.

As already mentioned above, it is preferred that the second material is biocompatible, what is particularly relevant for materials, with which humans, animals, or a sensitive environment can get in contact. Biocompatibility is exceptionally relevant for medical devices that are developed to be implanted into humans or animals, and the materials used for such devices should pass the mandatory biocompatibility and toxicity tests, e.g. the biocompatibility test according to DIN EN ISO 10993. The requirements concerning biocompatibility also hold for the first material, if this can get in contact with humans, animals, or a sensitive environment.

Erodable materials according to the invention include materials that are ablated or get brittle or fragile on continued exposure to an external stimulus. The external stimulus can be e.g. ambient air, exhaust fumes, specific gases, light, in particular UV light, high energy radiation like e.g. X-rays, alpha, beta or gamma rays, heat, smoke, water, e.g. waste water, solvents, microbes, et cetera. The external stimulus can also be a mechanical impact like fine particles in the passing gas, air or liquid, rubbing contact to a surface, et cetera. In this embodiment the second material is selected from materials that are known to be erodible in the aforementioned sense for a given external stimulus. In a predefined time of exposure to said external stimulus, that is effective integrally, the second material will be eroded to such an extent, that the first material is released and changes back to its permanent form. The time for this erosion varies with the surface, the mass and the chemical composition of the used material and can easily be adjusted by usual experiments. A device of the invention with such a second material can advantageously be used as a sensor or an actor (e.g. switch, valve), that detects a predefined integral magnitude (e.g. amount or dose) of an external stimulus and indicates this by a change in shape of the first material, wherein this change in shape can also be used to actuate a mechanical, electromechanical, optomechanical, et cetera, device like a switch or a valve. Thereby e.g. an electrical or optical signal can be switched on or off, that is recognized by a human being, or that is processed and evaluated automatically by an electric or electronic device, in particular a device including a microcontroller or a microprocessor like a computer. Alternatively or in addition a valve or flap in their broadest sense can directly be actuated by the first material when recovering its memorized shape. This allows e.g. a direct reaction in the case of an emergency situation like an open fire or radioactive fallout, without the need of any electrical components and without the need of a human action.

If more than one of the devices described above are used, that differ in the erodible material, it is possible to detect several stimuli at the same time, if appropriate second materials are selected as described above. If in such devices the second material is eroded by the same stimulus, but to a different extend, than different levels of integral exposure to the stimulus can be detected. Such an assembly of different devices can also be replaced by one device, if the first material is made as one-piece, that is divided into different zones, wherein the zones can at least partly recover their permanent form independently from the other zones, and the zones are hold in their temporary form by embodiments of the second material that differ as described above, in that they are sensitive to different stimuli and/or have a different sensitivity to the same stimulus. In this way very versatile and compact sensors and/or actuators can be designed.

Such sensors and actuators of the invention can particularly advantageously be used as fire and smoke sensors as mentioned above, for environmental surveillance and protection of human beings and equipment. Examples for such uses are the detection of i) water pollution, ii) the corrosiveness of ambient air or e.g. cooling liquids, iii) an amount of UV or high energy radiation, gas or chemicals that is harmful for an organism like a human being, an animal or a plant, or for a technical equipment, iv) a microbic affection, v) unwanted products in a reaction mixture, gas or solvent, and vi) the amount of fine particles in the ambient air. The erosion by e.g. an equipment that is rubbing on the device of the invention, can e.g. be used to detect an excessive vibration of this equipment or to indicate its maximum reliability, if its only rubbing to the device of the invention when being operative under load.

A special group of the aforementioned erodible materials are materials which degrade, for example by hydrolytic or enzymatic processes. As described above, devices including such materials as second material can be used to detect the existence of such an environment qualitatively as well as quantitatively. A further advantage of this embodiment of the device of the invention is particularly relevant for medical devices, that are designed to be implanted into the human or animal body, as it allows that the second material is eroded after implantation. This erosion can be used to effect the release of the first material, but can also be used to only remove the second material from the place of implantation, if the second material has already lost its fixing-ability under the impact of a different stimulus.

The shape memory device in accordance with the present invention may be prepared in a conventional manner using the forming techniques described in the art for shape memory polymers. The first material has to be provided in the original i.e. permanent shape for the desired article of manufacture. Subsequently the article is deformed until the desired deformed shape is obtained, which in turn then is fixed using the second material, for example by applying a partial or complete coating using conventional techniques. The article obtained accordingly is fixed in the deformed shape and the original shape can only be recovered by applying an external stimulus as indicated above.

The shape memory devices in accordance with the present invention may in particular be used in applications where a change in shape in response to an external mechanical force is suitable, for example in pressure sensors. Other applications are areas where a shape change in response to a tensile force or compression force is desired, for example sensors, but also medical devices as well as other articles of manufacture, such as toys etc. Further fields of application are medical devices, such as in particular stents, which may be fixed in a compressed, i.e. small diameter shape, by the second material, which in turn then is softened or removed after insertion so that the stent recovers its original shape

What is claimed is:

1. A shape memory device, comprising a first material, able to memorize an original shape and being present in a deformed shape, and a second material, fixing the first material in the deformed shape, wherein the second material looses its ability to fix the first material in the deformed shape upon application of an external stimulus.

2. The shape memory device of claim 1, wherein the external stimulus is a mechanical compression.

3. The shape memory device of claim 1, wherein the second material partially or completely covers the first material.

4. The shape memory device of claim 1, wherein the second material upon application of an organic solvent has a physical state selected from the group consisting of brittle, loss of integrity and loss of cohesion.

5. The shape memory device of claim 1, wherein the second material is susceptible to degradation by a means selected from the group consisting of hydrolytic and enzymatic.

6. The shape memory device any of claim 1, wherein the first material is selected from the group consisting of a shape memory polymer, natural rubber material, synthetic rubber material, and polymer network rubber material.

7. The shape memory device of claim 1, wherein the first material is a shape memory polymer present in an additional deformed shape.

8. The shape memory device any of claim 1, wherein the second material is a thermoplastic polymers.

9. The shape memory device of claim 1 wherein the device is selected from the group consisting of sensors, medical devices, stents, solvent detectors, valves, flaps, fire sensors, smoke sensors, devices that react to fire, devices that react to radioactivity, and devices that react to a predefined integral magnitude of the external stimulus.

10. (canceled)

11. The shape memory device of claim 1, wherein the second material is a light sensitive shape memory material.

12. The shape memory device of claim 1, wherein the external stimulus is a solvent.

13. The shape memory device of claim 1, wherein the external stimulus is light.

14. The shape memory device of claim 1, wherein the external stimulus is selected from the group consisting of heat sufficient to melt, heat sufficient to liquefy, and heat sufficient to sublimate the second material.

15. The shape memory device of claim 1, wherein the external stimulus is an ultrasonic frequency.

16. The shape memory device of claim 1, wherein the external stimulus is selected from the group consisting of exposure to chemicals that decrease the melting point and exposure to chemicals that decrease the viscosity of the second material.

17. The shape memory device of claim 1, wherein the external stimulus is a change in pH.

18. The shape memory device of claim 1, wherein the external stimulus is a magnetic field.

19. The shape memory device of claim 1, wherein the external stimulus is selected from the group consisting of causes ablation, increases brittleness and increases fragility of the second material.

20. The shape memory device of claim 19, wherein the external stimulus is selected from the group consisting of ambient air, exhaust fume, gas, light, UV light, high energy radiation, heat, smoke, water, waste water, solvent, microbe, mechanical impact, polluted water, corrosive ambient air, corrosive liquid, harmful chemical, reaction product in chemical synthesis, fine particle, and vibration.

21. The shape memory device of claim 1, wherein the second material is selected from the group consisting of a material in a crystalline state, semicrystalline state, water based material, pH sensitive material, light sensitive material, material with a non-harmful degradation product, biocompatible material, material sensitive to a magnetic field, and erodible material.

22. A method of using the shape memory device of claim 1, comprising the steps of:
a) inserting the shape memory device into a human body,
b) placing the shape memory device in a desired location within the human body,
c) applying an external stimulus, and
d) expanding the shape memory device into the original shape.
